(19) United States
(12) Patent Application Publication
(10) Pub. No.: US 2026/0176597 A1
(43) Pub. Date: Jun. 25, 2026

(54) MESSENGER RNA WITH HETEROLOGOUS UNTRANSLATED REGIONS FOR ENHANCED EXPRESSION AND USES THEREOF

(71) Applicant: Development Center For Biotechnology, New Taipei City (TW)

(72) Inventors: YI-YU KE, Taipei City (TW); Wan-Chen Hsieh, Taipei City (TW); Yu-Chi Li, Taipei City (TW); Chi-Tang Wang, Taipei City (TW); Feng-Peng Chang, Taipei City (TW); Tien-Hua Chu, Taipei City (TW)

(73) Assignee: Development Center for Biotechnology, New Taipei City (TW)

(21) Appl. No.: 19/429,554

(22) Filed: Dec. 22, 2025

Related U.S. Application Data
(60) Provisional application No. 63/738,106, filed on Dec. 23, 2024.

(51) Int. Cl. C12N 9/02; A61K 38/00; A61P 1/16; C12N 9/90; C12P 19/34

(52) U.S. Cl. CPC C12N 9/0071; A61P 1/16; C12N 9/90; C12P 19/34; C12Y 114/16001; C12Y 504/99002; A61K 38/00

(57) ABSTRACT

Disclosed herein are a messenger RNA (mRNA) comprising an open reading frame (ORF) encoding a protein of interest, a heterologous 5′ untranslated region (UTR) and/or a heterologous 3′ UTR for highly expressing the protein of interest. A method for synthesizing the mRNA and uses of the mRNA are also provided.

### PRIORITY INFORMATION

The subject application claims priority to and benefit of U.S. Provisional Patent Application No. 63/738,106, filed Dec. 23, 2024, the content of which is incorporated herein by reference in its entirety.

### REFERENCE TO ELECTRONIC SEQUENCE LISTING

The instant application contains a Sequence Listing which is submitted electronically in .xml format. The .xml copy was created on Dec. 11, 2025, is named “PC0802.xml” and is 34,000 bytes in size. The Sequence Listing contained in this .xml file is part of the specification and is hereby incorporated by reference herein in its entirety.

### FIELD OF THE INVENTION

The present disclosure in general relates to recombinant gene expression; more particularly, to a messenger RNA (mRNA) with heterologous untranslated regions (UTR) for enhanced expression and uses thereof.

### BACKGROUND OF THE INVENTION

Messenger RNA is a vital molecule in the process of gene expression, serving as the intermediary between DNA and protein synthesis. It is synthesized in the nucleus of eukaryotic cells during transcription, where a specific segment of DNA is copied into a complementary RNA sequence. This mRNA then exits the nucleus and enters the cytoplasm, where it serves as a template for translation, the process by which ribosomes synthesize proteins by decoding the mRNA sequence into a chain of amino acids. mRNA plays a crucial role in conveying genetic information and regulating cellular functions, and its applications have expanded significantly in recent years, particularly in the development of mRNA vaccines, which harness this technology to elicit immune responses against pathogens.

Enhancing the expression of mRNA is needed in this field.

### SUMMARY OF THE INVENTION

As embodied and broadly described herein, an mRNA with heterologous untranslated regions for enhanced expression is provided.

Accordingly, the present disclosure provides a messenger RNA comprising an open reading frame (ORF) encoding a protein of interest, wherein the ORF is flanked by a heterologous 5′ UTR and/or a heterologous 3′ UTR, wherein the 5′ UTR and/or 3′ UTR is derived from alpha-1-microglobulin/bikunin precursor (AMBP) or human alpha-globin gene.

In some embodiments of the disclosure, the 5′ UTR comprises a nucleotide sequence selected from the group consisting of SEQ ID NOs: 3 and 1.

In some embodiments of the disclosure, the 3′ UTR comprises a nucleotide sequence selected from the group consisting of SEQ ID NOs: 2 and 4.

In some embodiments of the disclosure, the 5′ UTR comprises a nucleotide sequence of SEQ ID NO: 3 and the 3′ UTR comprises a nucleotide sequence of SEQ ID NO: 2;
the 5′ UTR comprises a nucleotide sequence of SEQ ID NO: 1 and the 3′ UTR comprises a nucleotide sequence of SEQ ID NO: 2;
the 5′ UTR comprises a nucleotide sequence of SEQ ID NO: 1 and the 3′ UTR comprises a nucleotide sequence of SEQ ID NO: 4; or
the 5′ UTR comprises a nucleotide sequence of SEQ ID NO: 3 and the 3′ UTR comprises a nucleotide sequence of SEQ ID NO: 4.

Examples of the protein of interest include, but are not limited to, phenylalanine hydroxylase (PAH), methylmalonyl-CoA mutase (MMUT), interferon alpha, TNF-related apoptosis-inducing ligand, vascular adhesion protein 1, hepatocyte growth factor, G6PC, ABCB11, ABCB4, ASL1, ASS, Arg1, AGXT, OTC, CBS, GBE, GALE, HADH, MCCC1, ADAMTS13, SLC25A15, C2, F2, PROS1, SERPINA1, GALT1, ETFA, GCDH, CTNS, FAH, TAT, HPD, HSD17b4, SLC2A2, GALC, ABCC6, AHSG, PDC-E2, GAA, ATP8B1, MMACHC, GK, PCCA, PCCB and UGT1A1 or fragments thereof. In some embodiments of the disclosure, the protein of interest is selected from the group consisting of SEQ ID NOs. 5, 6, 9, and 11 to 17.

In some embodiments of the disclosure, the mRNA further comprises a 5′ cap and/or a poly A tail.

In some embodiments of the disclosure, the mRNA comprises a nucleotide sequence selected from the group consisting of:
a combination of SEQ ID NOs. 3, 5, and 2;
a combination of SEQ ID NOs. 1, 5, and 4;
a combination of SEQ ID NOs. 1, 5, and 2;
a combination of SEQ ID NOs. 3, 13, and 2;
a combination of SEQ ID NOs. 3, 14, and 2;
a combination of SEQ ID NOs. 3, 15, and 2; and
a combination of SEQ ID NOs. 3, 16, and 2.

The present disclosure also relates to a DNA for transcripting the mRNA as described herein.

In some embodiments of the disclosure, the DNA further comprises a promoter. For example, the promoter is an RNA polymerase promoter.

The present disclosure also relates to a method for synthesizing the mRNA as described herein, which comprises
providing the DNA; and
conducting a transcription form the DNA.

In some embodiments of the disclosure, the method further comprises conducting a capping reaction.

The present disclosure also relates to a composition comprising the mRNA as described herein and a pharmaceutically acceptable carrier.

In some embodiments of the disclosure, the pharmaceutically acceptable carrier is a liposome or lipid nanoparticle.

The present disclosure also relates to a method for expressing a protein of interest in a cell comprising introducing the mRNA as described herein to the cell. For example, the cell is a liver cell.

The present disclosure also relates to a method for treating a liver disease in a subject in need of such treatment comprising administering the mRNA or the composition as described herein to the subject. Alternatively, the present disclosure also relates to use of the mRNA or the composition as described herein in the manufacture of a medicament for treating a liver disease in a subject in need of such treatment. Alternatively, the present disclosure also relates to the mRNA or the composition as described herein for use in treating a liver disease in a subject in need of such treatment.

Examples of the liver disease include, but are not limited to, phenylketonuria (PKU), methylmalonic aciduria (MMA), acute hepatitis, chronic hepatitis, liver cirrhosis, cirrhosis, fatty liver, liver cancer, glycogen storage disease, progressive familial intrahepatic cholestasis 1 (PFIC1), progressive familial intrahepatic cholestasis 2 (PFIC2), progressive familial intrahepatic cholestasis 3 (PFIC3), adenylosuccinate lyase deficiency (ASLD), citrullinemia, arginase-1 deficiency, primary hyperoxaluria type 1 (PH1), ornithine transcarbamylase deficiency (OTCD), homocystinuria, pheylketonuria, glycogen storage disease type IV (GSDIV), galactose-1-phosphate uridylyltransferase deficiency (type I (GALTI), type II (GALTII) or type 3 (GALTIII)), long chain 3-hydroxyacyl-CoA dehydrogenase deficiency (LCHADD), 3-methylcrotonyl-CoA carboxylase deficiency (MCCC1 deficiency), MMA and homocystinuria type C (MMACHC), thrombotic thrombocytopenic purpura (TTP), hyperornithinemia-hyperammonemia-homocitrullinuria syndrome (HHH), complement component 2 deficiency (C2D), F2, protein S deficiency (caused by allelic variants of PROS1), alpha-1 antitrypsin deficiency (A1AT), glutaric academia 1 (GA-1), glutaric academia 2 (GA-2), cystinosis (CTNS), tyrosinemia, tyrosinemia type 3 (cause by allelic variants of HPD), D-bifunctional protein deficiency (DBP), Fanconi-Bickel syndrome (FBS), pseudoxanthoma elasticum (PXE), primary biliary cirrhosis, Pompe disease, glycerol kinase deficiency (GKD), proprionic acidemia (PA) and Crigler-Najjar syndrome (CN1).

### BRIEF DESCRIPTION OF DRAWINGS

FIG. 1 shows a schematic diagram of the structure of the PAH mRNAs in the expression vector.

FIG. 2 shows results of comparing the expression levels of PAH protein of the Examples and Comparative Examples.

FIG. 3 shows results of comparing the purity of PAH mRNA of the Examples and Comparative Examples.

FIG. 4 shows results of comparing the PAH activity of the Examples and Comparative Examples (PAH002 and MD202), compared to activity in control animals injected with PBS, as measured in blood phenylalanine levels over time.

FIG. 5 shows a schematic diagram of the structure of the MUT mRNAs in the expression vector.

FIG. 6 shows results of the two experiments comparing the expression levels of MUT protein of the Examples and Comparative Examples.

FIG. 7 shows Results of the two experiments comparing the dsRNA (%) of MUT mRNA of the Examples and Comparative Examples.

### DETAILED DESCRIPTION OF THE INVENTION

Unless otherwise defined, scientific and technical terms used herein shall have the meanings that are commonly understood by those of ordinary skill in the art. Furthermore, unless otherwise required by context, singular terms shall include pluralities and plural terms shall include the singular. Generally, nomenclatures used in connection with, and techniques of, cell and tissue culture, molecular biology, and protein and oligo- or polynucleotide chemistry and hybridization described herein are those well-known and commonly used in the art.

The practice of the present disclosure may employ technologies comprising conventional techniques of cell biology, cell culture, antibody technology, and genetic engineering, which are within the ordinary skills of the art. Such techniques are explained fully in the literature.

As utilized in accordance with the present disclosure, the following terms, unless otherwise indicated, shall be understood to have the following meanings: The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

It must be noted that, as used in this specification and the appended claims, the singular forms “a,” “an,” and “the” include plural referents unless the content clearly dictates otherwise.

As used herein, the term “messenger RNA” (mRNA) refers to any polynucleotide which encodes a protein of interest and which is capable of being translated to produce the encoded protein of interest in vitro, in vivo, in situ or ex vivo.

As used herein, “open reading frame” or “ORF” refers to a length of RNA or DNA sequence, between a translation start signal (e.g., AUG or ATG, respectively) and any one or more of the known termination codons, which encodes one or more polypeptide sequences. Put another way, the ORF describes the frame of reference as seen from the point of view of a ribosome translating the RNA code, insofar that the ribosome is able to keep reading (i.e., adding amino acids to the nascent protein) because it has not encountered a stop codon. Thus, “open reading frame” or “ORF” refers to the amino acid sequence encoded between translation initiation and termination codons of a coding sequence. Here, the terms “initiation codon” and “termination codon” refer to a unit of three adjacent nucleotides (i.e., a codon) in a coding sequence that specifies initiation and chain termination, respectively, of protein synthesis (mRNA translation).

As used herein, the terms “polypeptide”, “protein”, and “peptide”, which are used interchangeably herein, refer to a polymer of the 20 protein amino acids, or amino acid analogs, regardless of its size or function. As used herein, the terms refer to a gene product. The term “polypeptide” encompasses proteins of all functions, including enzymes. Thus, exemplary polypeptides include gene products, naturally occurring proteins, homologs, orthologs, paralogs, fragments, and other equivalents, variants and analogs of the foregoing.

The term “heterologous” refers to a molecule or activity that is from a source that is different than the referenced molecule or organism. For example, in the context of the disclosure, a “heterologous 5′ UTR and/or 3′ UTR” refers to a 5′ UTR and/or 3′ UTR that is different from the referenced mRNA or gene. Therefore, when a 5′ UTR and/or 3′ UTR is removed from a gene and replaced with a 5′ UTR and/or 3′ UTR from a different gene, the modified gene has a “heterologous 5′ UTR and/or 3′ UTR.”

As used herein, the term “transcription” refers to a cellular process involving the interaction of an RNA polymerase with a gene that directs the expression as RNA of the structural information present in the coding sequences of the gene. The process includes, but is not limited to, the following steps: (a) the transcription initiation; (b) transcript elongation; (c) transcript splicing; (d) transcript capping; (e) transcript termination; (f) transcript polyadenylation; (g) nuclear export of the transcript; (h) transcript editing; and (i) stabilizing the transcript.

As used in the present disclosure, the term “pharmaceutical composition” refers to a mixture containing a therapeutic agent administered to a mammal, for example a human, for preventing, treating, or eliminating a particular disease or pathological condition that the mammal suffers.

As used herein, the terms “treatment,” “treating,” and the like, cover any treatment of a disease in a mammal, particularly in a human, and include: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As interchangeably used herein, the terms “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “in need of treatment” refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the compounds of the present disclosure.

The present disclosure provides a messenger RNA comprising an open reading frame encoding a protein of interest, wherein the ORF is flanked by a heterologous 5′ UTR and/or a heterologous 3′ UTR, wherein the 5′ UTR and/or 3′ UTR is derived from AMBP or human alpha-globin gene.

Traditionally, the basic components of an mRNA molecule include at least a coding region, a 5′ UTR, a 3′ UTR, a 5′ cap and a poly-A tail. The 5′ and 3′ UTRs, whose functions are related, respectively, regulate translation and maintain mRNA stability. The 5′ UTR is mainly involved in translation of its downstream ORF sequence. The Kozak sequence is generally added after the 5′ UTR sequence to improve translation efficiency. Conversely, the function of the 3′ UTR is to maintain mRNA stability. Studies have shown that adenylate-uridylate-rich elements are involved in mRNA degradation. Degradation rate and translation life cycle can be adjusted by replacing adenylate-uridylate-rich sequences found in the 3′ UTR. Design of proper 5′ and 3′ UTRs sequences is crucial for the success of mRNA vaccines. Many investigations have been conducted to screen and design the most effective 5′ and 3′ UTR sequences for mRNA vaccines, therefore UTR sequences are considered intellectual properties of vaccine manufacturers.

In some embodiments of the disclosure, the 5′ UTR derived from AMBP comprises a nucleotide sequence of SEQ ID NO: 1 or a variation thereof having sequence identity in at least 81%, 82%, 83%, 84%, 85%, 86%, 87%, 88%, 89%, 90%, 91%, 92%, 93%, 94%, 95%, 96%, 97%, 98%, 99%, or 100%.

In some embodiments of the disclosure, the 5′ UTR derived from a part of human alpha-globin comprises a nucleotide sequence of SEQ ID NO: 3 or a variation thereof having sequence identity in at least 81%, 82%, 83%, 84%, 85%, 86%, 87%, 88%, 89%, 90%, 91%, 92%, 93%, 94%, 95%, 96%, 97%, 98%, 99%, or 100%.

In some embodiments of the disclosure, the 3′ UTR derived from AMBP comprises a nucleotide sequence of SEQ ID NO: 2 or a variation thereof having sequence identity in at least 81%, 82%, 83%, 84%, 85%, 86%, 87%, 88%, 89%, 90%, 91%, 92%, 93%, 94%, 95%, 96%, 97%, 98%, 99%, or 100%.

In some embodiments of the disclosure, the 3′ UTR derived from a part of human alpha-globin comprises a nucleotide sequence of SEQ ID NO: 4 or a variation thereof having sequence identity in at least 81%, 82%, 83%, 84%, 85%, 86%, 87%, 88%, 89%, 90%, 91%, 92%, 93%, 94%, 95%, 96%, 97%, 98%, 99%, or 100%.

In some embodiments of the disclosure, the 5′ UTR comprises a nucleotide sequence of SEQ ID NO: 1 and the 3′ UTR comprises a nucleotide sequence of SEQ ID NO: 2. In some embodiments of the disclosure, the 5′ UTR comprises a nucleotide sequence of SEQ ID NO: 1 and the 3′ UTR comprises a nucleotide sequence of SEQ ID NO: 4. In some embodiments of the disclosure, the 5′ UTR comprises a nucleotide sequence of SEQ ID NO: 3 and the 3′ UTR comprises a nucleotide sequence of SEQ ID NO: 2. In some embodiments of the disclosure, the 5′ UTR comprises a nucleotide sequence of SEQ ID NO: 3 and the 3′ UTR comprises a nucleotide sequence of SEQ ID NO: 4.

In some embodiments, the mRNA according to the disclosure is utilized for expression a gene for exhibiting liver functions. In some embodiments, the protein of interest is phenylalanine hydroxylase. In some embodiments, codons encoding the protein of interest is optimized. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis.

Examples of the protein of interest include, but are not limited to, phenylalanine hydroxylase (PAH), methylmalonyl-CoA mutase (MMUT), interferon alpha, TNF-related apoptosis-inducing ligand, vascular adhesion protein 1, hepatocyte growth factor, G6PC, ABCB11, ABCB4, ASL1, ASS, Arg1, AGXT, OTC, CBS, GBE, GALE, HADH, MCCC1, MUT, ADAMTS13, SLC25A15, C2, F2, PROS1, SERPINA1, GALT1, ETFA, GCDH, CTNS, FAH, TAT, HPD, HSD17b4, SLC2A2, GALC, ABCC6, AHSG, PDC-E2, GAA, ATP8B1, MMACHC, GK, PCCA, PCCB and UGT1A1 or fragments thereof.

In some embodiments of the disclosure, the PAH is encoded by the nucleotide having the sequence of SEQ ID NOs. 5, 6, 9, 11 and 12, particularly SEQ ID NOs. 5, 6, and 12 or a variation thereof having sequence identity in at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In some embodiments of the disclosure, the mRNA comprises a nucleotide sequence selected from the group consisting of:
a combination of SEQ ID NOs. 3, 5, and 2;
a combination of SEQ ID NOs. 1, 5, and 4; and
a combination of SEQ ID NOs. 1, 5, and 2.

In some embodiments of the disclosure, the MMUT is encoded by the nucleotide having the sequence of SEQ ID NOs. 13 to 17, particularly SEQ ID NOs. 13 to 16 or a variation thereof having sequence identity in at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In some embodiments of the disclosure, the mRNA comprises a nucleotide sequence selected from the group consisting of:
a combination of SEQ ID NOs. 3, 13, and 2;
a combination of SEQ ID NOs. 3, 14, and 2;
a combination of SEQ ID NOs. 3, 15, and 2; and
a combination of SEQ ID NOs. 3, 16, and 2.

In some embodiments of the disclosure, the mRNA further comprises a 5′ cap and/or a poly A tail.

Eukaryotic messenger RNAs (mRNAs) carry a specific structure at the 5′-end, the so-called cap structure. This consists of a N7-methylated guanosine moiety, which is added to the first transcribed nucleotide of an RNA, commonly a guanosine, via a 5′-5′ triphosphate bridge. Capped mRNAs can be obtained by in vitro transcription by adding an excess of a cap dinucleotide, e.g., m7GpppG, to the reaction. In another aspect, the poly A tail sequence of RNA is important for nuclear export, RNA stability and translational efficiency of eukaryotic mRNA. The poly A tail sequence is shortened over time and if short enough, the RNA is degraded enzymatically.

The present disclosure also relates to a DNA for transcripting the mRNA as described herein. The DNA is known as a DNA template which can be in a form of plasmid or vector. In some embodiments of the disclosure, the DNA further comprises a promoter. For example, the promoter is an RNA polymerase promoter.

The present disclosure also relates to a method for synthesizing the mRNA as described herein, which comprises
providing the DNA; and
conducting a transcription form the DNA.

The process of mRNA production may include, but is not limited to, in vitro transcription, cDNA template removal and RNA clean-up, and mRNA capping and/or tailing reactions.

The DNA may be transcribed using an in vitro transcription (IVT) system. The system typically comprises a transcription buffer, nucleotide triphosphates (NTPs), an RNase inhibitor and a polymerase. The NTPs may be manufactured in house, may be selected from a supplier, or may be synthesized as described herein. The NTPs may be selected from, but are not limited to, those described herein including natural and unnatural (modified) NTPs. The polymerase may be selected from, but is not limited to, T7 RNA polymerase, T3 RNA polymerase and mutant polymerases.

Cellular transcription is couple to 5′ capping and other mRNA post-transcriptional processing (such as, but not limited to, splicing and polyadenylation) in various systems. Using a chimeric enzyme comprising both a polymerase and a capping enzyme could allow for high efficiency 5′ capping of the mRNA of the present disclosure.

The present disclosure also relates to a composition comprising the mRNA as described herein and a pharmaceutically acceptable carrier. Particularly, the composition is a pharmaceutical composition.

In some embodiments of the disclosure, the pharmaceutically acceptable carrier is a liposome or lipid nanoparticle. Lipid encapsulated mRNA formulations, such as lipid nanoparticle (LNP) compositions show high degree of cellular uptake and protein expression. Lipid nanoparticle formulations traditionally use ethanol as a solvent for the lipid solution which is then mixed with an mRNA solution.

Compositions formed via the methods described herein may be particularly useful for administering an agent to a subject in need thereof. In some embodiments, the compositions are used to deliver a pharmaceutically active agent. The compositions may be administered in any way known in the art of drug delivery, for example, orally, parenterally, intravenously, intramuscularly, subcutaneously, intradermally, transdermally, intrathecally, submucosally, sublingually, rectally, vaginally, etc.

Once the compositions have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions described herein and for use in accordance with the embodiments described herein may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

The present disclosure also relates to a method for expressing a protein of interest in a cell comprising introducing the mRNA as described herein to the cell. For example, the cell is a liver cell.

mRNA gene therapy can fundamentally address many liver diseases. The mRNA as described herein has high stability and can extend its duration in the body. By optimizing the codons of the mRNA and adding a 5′ cap structure and poly A tail, the stability of the mRNA and its translation efficiency within cells can be significantly enhanced. Choosing the appropriate combinations of 5′ and 3′ UTRs is crucial for the translation of mRNA in liver cells. The disclosure identifies mRNA drug combinations that can achieve high expression in liver cells through the optimization of codons and the combination of UTRs.

The present disclosure also relates to a method for treating a liver disease in a subject in need of such treatment comprising administering the mRNA or the composition as described herein to the subject. Alternatively, the present disclosure also relates to use of the mRNA or the composition as described herein in the manufacture of a medicament for treating a liver disease in a subject in need of such treatment. Alternatively, the present disclosure also relates to the mRNA or the composition as described herein for use in treating a liver disease in a subject in need of such treatment.

Examples of the liver disease include, but are not limited to, phenylketonuria (PKU), methylmalonic aciduria (MMA), acute hepatitis, chronic hepatitis, liver cirrhosis, cirrhosis, fatty liver, liver cancer, glycogen storage disease, progressive familial intrahepatic cholestasis 1 (PFIC1), progressive familial intrahepatic cholestasis 2 (PFIC2), progressive familial intrahepatic cholestasis 3 (PFIC3), adenylosuccinate lyase deficiency (ASLD), citrullinemia, arginase-1 deficiency, primary hyperoxaluria type 1 (PH1), ornithine transcarbamylase deficiency (OTCD), homocystinuria, pheylketonuria, glycogen storage disease type IV (GSDIV), galactose-1-phosphate uridylyltransferase deficiency (type I (GALTI), type II (GALTII) or type 3 (GALTIII)), long chain 3-hydroxyacyl-CoA dehydrogenase deficiency (LCHADD), 3-methylcrotonyl-CoA carboxylase deficiency (MCCC1 deficiency), MMA and homocystinuria type C (MMACHC), thrombotic thrombocytopenic purpura (TTP), hyperornithinemia-hyperammonemia-homocitrullinuria syndrome (HHH), complement component 2 deficiency (C2D), F2, protein S deficiency (caused by allelic variants of PROS1), alpha-1 antitrypsin deficiency (A1AT), glutaric academia 1 (GA-1), glutaric academia 2 (GA-2), cystinosis (CTNS), tyrosinemia, tyrosinemia type 3 (cause by allelic variants of HPD), D-bifunctional protein deficiency (DBP), Fanconi-Bickel syndrome (FBS), pseudoxanthoma elasticum (PXE), primary biliary cirrhosis, Pompe disease, glycerol kinase deficiency (GKD), proprionic acidemia (PA) and Crigler-Najjar syndrome (CN1).

In some embodiments, the liver disease is phenylketonuria. Phenylketonuria (PKU), caused by a deficiency of phenylalanine hydroxylase (PAH), is a recessive hereditary metabolic disorder. The vast majority of cases arise from individuals lacking the enzyme that metabolizes phenylalanine (Phe), or from reduced activity or insufficient production of this enzyme due to genetic mutations. This leads to excessive accumulation of phenylalanine from dietary sources, which can result in intellectual disabilities, seizures, and mental disorders in patients. Additionally, the body may produce a musty odor or exhibit lighter skin pigmentation.

The main goal of treating PKU is to lower the concentration of phenylalanine (Phe) in the body to prevent neurological damage. Common treatment methods include lifelong dietary control, BH4 (tetrahydrobiopterin) supplementation therapy, large neutral amino acid (LNAA) supplementation, and enzyme replacement therapy. mRNA gene therapy is currently under research, aiming to deliver a healthy PAH gene to repair or replace the defective PAH gene, thereby promoting normal enzyme expression. The method according to the disclosure could fundamentally address the cause of PKU without permanently altering the patient's genomic structure, offering higher safety and flexibility, and potentially improving the patient's quality of life.

In some embodiments, the liver disease is methylmalonic acidemia. Patients with MMA are unable to properly metabolize certain parts of proteins and fats due to a deficiency of methylmalonyl-CoA mutase (MMUT), leading to the accumulation of methylmalonic acid in the body. This is a hereditary metabolic disorder. The MMUT enzyme normally breaks down methylmalonic acid, but when MMUT function is abnormal, methylmalonic acid accumulates in large amounts, causing a series of metabolic disturbances. The conventional treatments for MMA include vitamin B12 supplementation (effective for some patients), dietary control (low-protein diet, special formula milk), and drug therapy (such as carnitine and amino acid chelators). The mRNA or composition as disclosed herein aims to deliver a healthy MMUT gene to repair or replace the defective MMUT gene, thereby promoting normal enzyme expression. In some embodiments, the method as disclosed herein can fundamentally address the cause of MMA without permanently altering the patient's genomic structure, providing higher safety and flexibility, and potentially improving the patient's quality of life.

The following examples are provided to aid those skilled in the art in practicing the present disclosure.

### EXAMPLES

### Example 1 mRNA for PAH

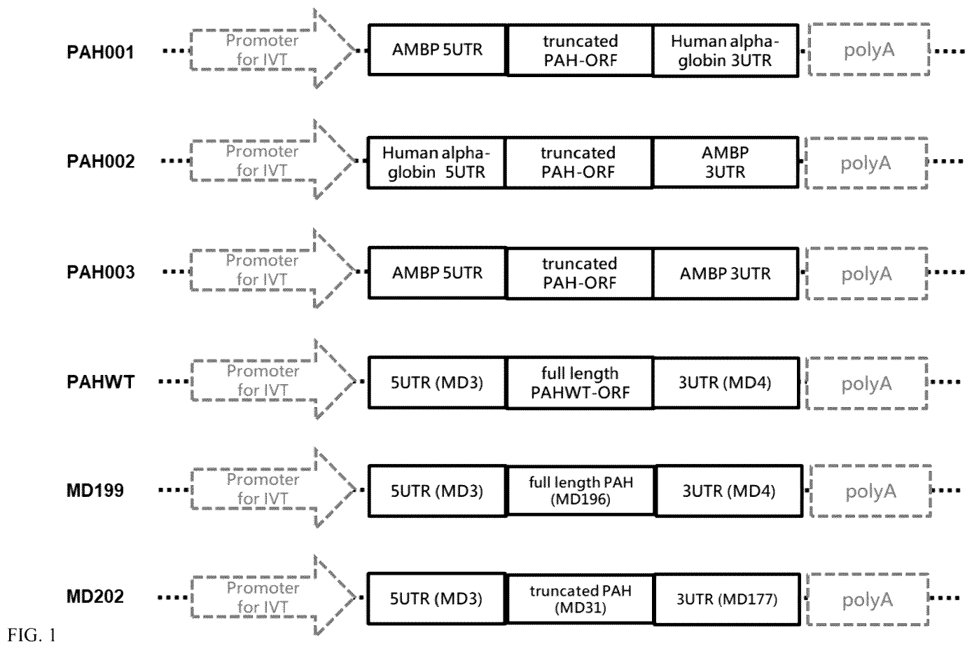

The CDS codons were optimized for truncated WT PAH, and combined with the 3′ UTRs and 5′ UTRs of liver tissue highly expressed genes respectively. The AMBP 5UTR is SEQ ID NO: 1; the AMBP 3UTR is SEQ ID NO: 2; the human alpha-globin 5UTR is SEQ ID NO: 3; the human alpha-globin 3UTR is SEQ ID NO: 4; the ORF of truncated PAH (truncated PAH-ORF) is SEQ ID NO: 5; the open reading frame (ORF) of full-length (FL) wildtype PAH (full length PAHWT-ORF) is SEQ ID NO: 6; the 5UTR of MD3 is SEQ ID NO: 7; the 3UTR of MD4 is SEQ ID NO: 8; the truncated PAH (MD31) is SEQ ID NO: 9; the 3UTR of MD177 is SEQ ID NO: 10; the full length PAH (MD196) is SEQ ID NO: 11; and the ORF of truncated PAHWT (truncated PAHWT-ORF) is SEQ ID NO: 12. These were then combined to generate Examples PAH001, PAH002, and PAH003. The UTR combinations of MD3 and MD4 with full-length PAH and truncated PAH were also formed as Comparative Examples of MD199 and MD202 (FIG. 1).

### DNA Template Synthesis

Plasmids containing nucleic acid sequences designed with different genes of interest (GOI) were transfected into bacteria. These bacteria are then cultured and amplified in LB medium containing antibiotics, followed by concentration and purification of the plasmid DNA. The purified DNA was subsequently linearized using restriction enzymes, and after further concentration and purification, the quality of the linearized DNA template was assessed.

### In Vitro Transcription (IVT) for Synthesis

In Vitro Transcription was performed to produce Pre-mRNA. Using T7 polymerase enzyme, PPase, RNase inhibitor, Mg²⁺, and 10× reaction buffer mixed with a linearized DNA template, in vitro transcription (IVT) was carried out to synthesize mRNA. After initial purification with LiCl, enzymes were used to carry out the mRNA capping reaction, and finally purify and recover the mRNA using LiCl.

Quality Control of the In Vitro Transcribed mRNA

NanoDrop™ was used to measure the 260/280 and 260/230 ratios for assessing yield and quality, while gel electrophoresis was employed to evaluate sample purity. Capillary electrophoresis was utilized to analyze the integrity of the samples, and Mass Spectrometry was conducted to determine capping efficiency. Additionally, an ELISA-based J2 antibody assay was performed to detect double-stranded RNA (dsRNA).

The quality of mRNA is shown in Table 1.

TABLE 1

| | | | | | Total | | | |
|---|---|---|---|---|---|---|---|---|
| | | | | Conc. | mass | Productivity | | Capping |
| | sample | 260/280 | 260/230 | (ng/ml) | (mg) | (mg/ml) | CE (%) | (%) |
| #1 | PAH002 | 1.953 | 2.375 | 1056.15 | 211.23 | 10.56 | 88.6% | 100% |
| | MD202 | 1.833 | 2.222 | 586.88 | 117.376 | 5.87 | 80.1% | 86% |
| #2 | PAH002 | 1.95 | 2.321 | 903.7 | 180.74 | 9.04 | 91.5% | 96.3% |
| | MD202 | 1.87 | 2.196 | 655.48 | 65.548 | 3.28 | 79.9% | 91.7 |

Cell Culture PAH mRNA Transfections for PAH Protein Expression

The prepared mRNA was transfected into Hep3B cells. After 24 hours, the cells were lyzed using RIPA buffer. The total cell concentration was measured, and the expression level of human PAH protein was analyze using Western blotting.

Synthesis of mRNA LNP

The LNP preparation method involves dissolving ionizable lipids and other lipid components in ethanol. The mixture was then rapidly mixed with a citrate buffer solution containing the nucleic acid (PAH002 or MD202) via a microfluidic device, allowing the lipids to self-assemble into mRNA-LNPs encapsulating the nucleic acid. Ultra Centrifugal Filters™ were then used to remove the ethanol, followed by solvent exchange and concentration with PBS buffer. Nucleic acid concentration was quantified using a Ribogreen™ assay.

### Codon-Optimized PAH and UTR Combinations

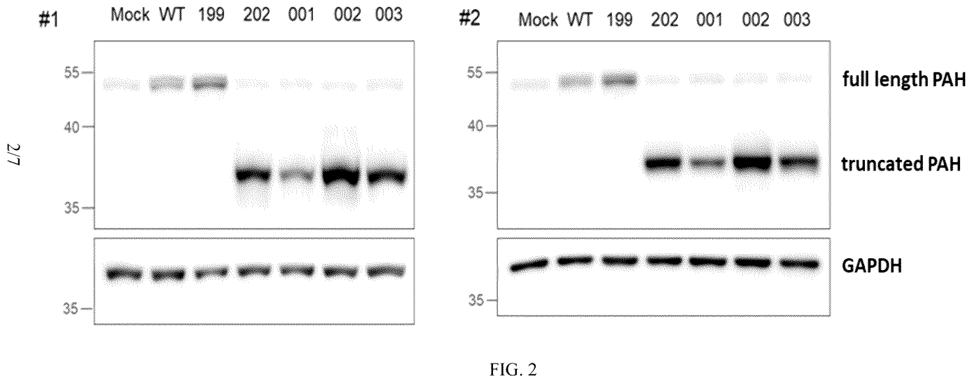

The Examples of PAH001, 002, and 003 were prepared and compared with the Comparative Examples of PAHWT, MD199, and MD202 as shown in FIG. 1. After DNA template generation, mRNA was produced using in vitro transcription (IVT), and then transfected into Hep3B cells. Protein expression was analyzed using Western blot. The results (FIG. 2) show that the protein expression level of the UTR combination PAH002 is significantly better than that of PAHWT, MD199 and MD202.

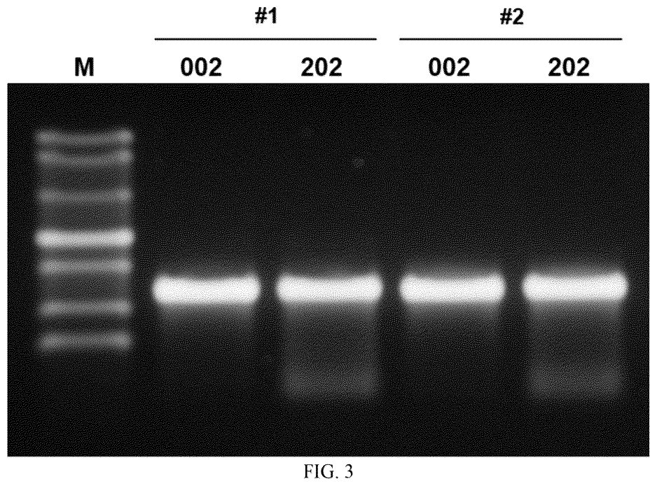

Under the same IVT and purification conditions, PAH002 and MD202 demonstrated superior mRNA quality in the concentration (ng/ml), total mass (mg), productivity (mg/ml), CE (%), and capping (%) (Table 1). Additionally, the gel image shows that the byproducts of PAH002 are significantly fewer than those of MD202 (FIG. 3), indicating that PAH002 promotes better RNA quality.

### Animal Models

To determine whether the PAH mRNA confers a therapeutic benefit in vivo, a PKU disease mouse model was applied to compare the efficacy of PAH-002 and the competitor MD202 mRNA under the same LNP encapsulation conditions. Blood samples were collected before administration (0 h) and at 6 h, 24 h, 48 h, 72 h, and 168 h after administration, and the phenylalanine concentration in the blood was analyzed by LC-MS/MS.

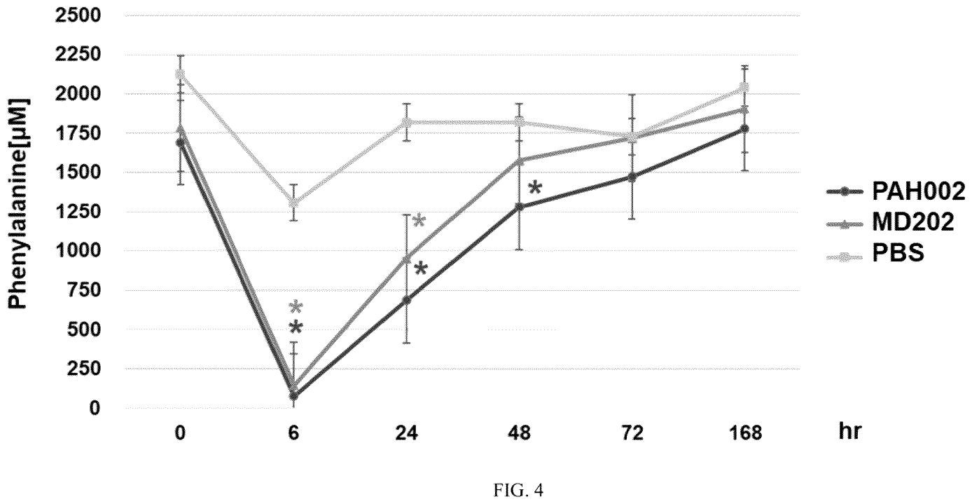

The results show that compared with the PBS group, PAH002 significantly reduced blood phenylalanine concentrations at 6, 24, and 48 hours, while MD202 only significantly reduced blood phenylalanine concentrations at 6 and 24 hours. PAH activity in the PAH002 group is higher than that in both the PBS and MD202 groups at all time points (FIG. 4).

### Example 2 mRNA for MMUT

The UTR combination of PAH002 was selected for MMUT. The methods applied in Example 2 is similar to those in Example 1.

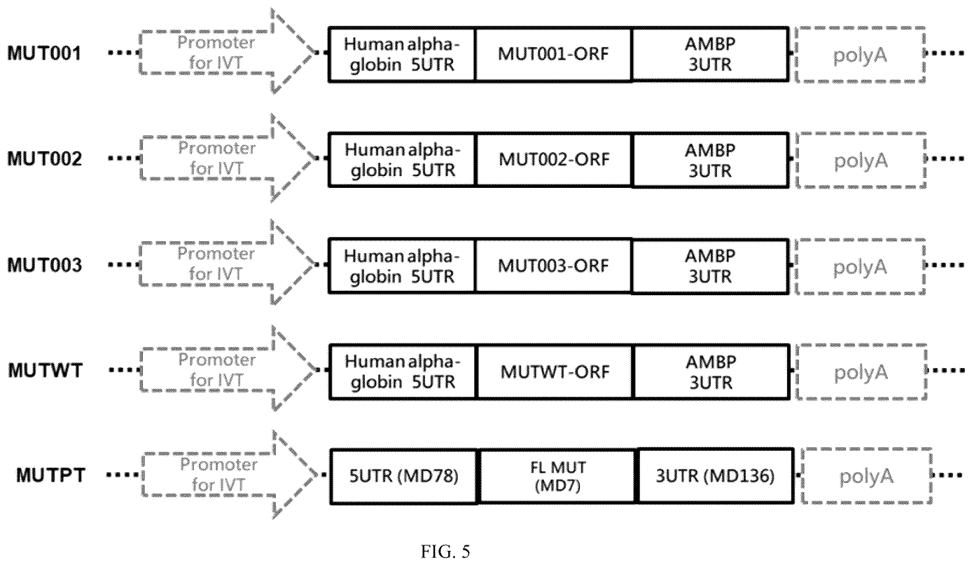

The CDS codons for WT MMUT (MUT) were optimized, and combined with the human alpha-globin 5′UTR and AMBP 3′UTR and further generated as Examples MUT001, MUT002, and MUT003. The MUT001-ORF is SEQ ID NO: 13; the MUT002-ORF is SEQ ID NO: 14; the MUT003-ORF is SEQ ID NO: 15; the MUTWT-ORF is SEQ ID NO: 16; the full length (FL) MUT (MD7) is SEQ ID NO: 17; the 5UTR (MD78) is SEQ ID NO: 18; and 3UTR (MD136) is SEQ ID NO: 19. The UTR combinations of MD78 and MD136 with full-length MUT were also formed as Comparative Examples of MUTPT (FIG. 5).

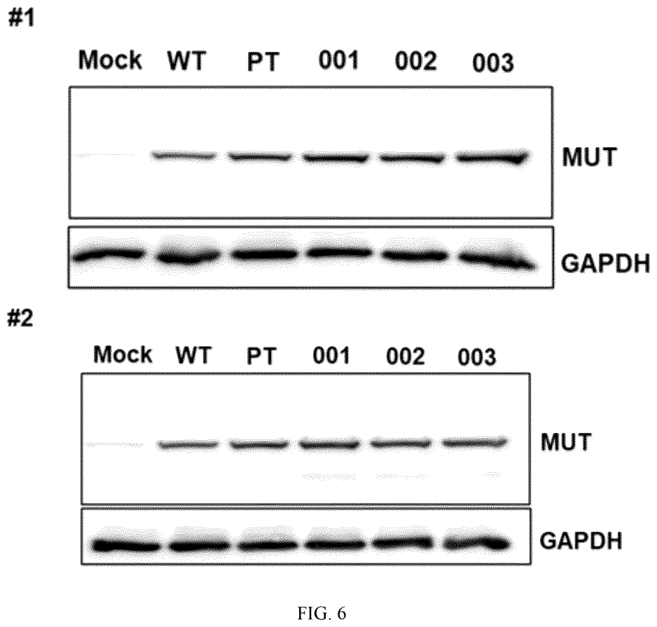

The Examples of MUT001, 002, and 003 were prepared and compared with the Comparative Examples of MUTPT as shown in FIG. 4. After DNA template generation, mRNA was produced using in vitro transcription (IVT), and then transfected into Hep3B cells. Protein expression was analyzed using Western blot. The results of the two experiments show that the protein expression levels of MUTPT, MUT001, MUT002, and MUT003 were slightly increased compared with the WT group (FIG. 6).

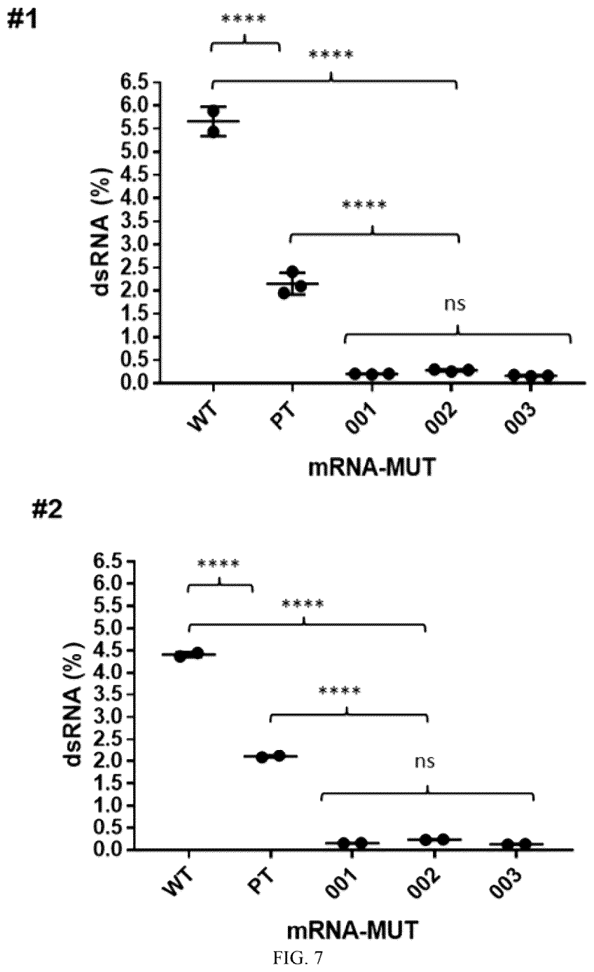

Under the same IVT and purification conditions, MUT001, MUT002, and MUT003 demonstrate superior mRNA quality in the concentration (ng/ml), total mass (mg), productivity (mg/ml), CE (%), and capping (%), and the byproducts dsRNA (ng/μg, and ratio) (Table 2) to MUTWT and MUTPT (FIG. 7).

The quality of mRNA is shown in Table 2.

TABLE 2

| | | | Conc. | Total mess | Productivity | CE | Capping | DsRNA | DsRNA |
|---|---|---|---|---|---|---|---|---|---|
| #1 | 260/280 | 260/230 | [ng/ml] | (mg) | (mg/ml) | (%) | (%) | (ng/ug) | (%) |
| MUTWT | 2.33 | 2.78 | 830.02 | 132.80 | 6.64 | 82.3 | 97.48% | 56.57 | 5.66% |
| MUTPT | 2.16 | 2.40 | 946.90 | 151.50 | 7.58 | 86.0 | 90.92% | 21.81 | 2.18% |
| MUT001 | 2.11 | 2.37 | 761.46 | 121.83 | 6.09 | 84.5 | 94.93% | 2.04 | 0.2% |
| MUT002 | 2.26 | 2.60 | 1154.70 | 92.38 | 4.62 | 83.2 | 93.73% | 2.73 | 0.27% |
| MUT003 | 2.13 | 2.36 | 888.40 | 142.14 | 7.11 | 84.1 | 95.88% | 1.55 | 0.16% |
| | | | Conc. | Total mess | Productivity | CE | Capping | DsRNA | DsRNA |
| #2 | 260/280 | 260/230 | [ng/ml] | (mg) | (mg/ml) | (%) | (%) | (ng/ug) | (%) |
| MUTWT | 2.32 | 2.79 | 918.70 | 275.61 | 5.51 | 85.5 | 97.35 | 44.04 | 4.4 |
| MUTPT | 2.13 | 2.37 | 954.84 | 381.94 | 7.64 | 82.4 | 98.90 | 21.08 | 2.1 |
| MUT001 | 2.18 | 2.41 | 1131.00 | 339.30 | 6.79 | 83.1 | 98.52 | 1.50 | 1.5 |
| MUT002 | 2.18 | 2.55 | 854.58 | 213.65 | 4.27 | 84.6 | 97.69 | 2.32 | 2.3 |
| MUT003 | 2.17 | 2.38 | 1098.00 | 329.40 | 6.59 | 83.0 | 98.96 | 1.28 | 1.3 |

While the present invention has been described in conjunction with the specific embodiments set forth above, many alternatives thereto and modifications and variations thereof will be apparent to those of ordinary skill in the art. All such alternatives, modifications and variations are regarded as falling within the scope of the present invention.

1. A messenger RNA (mRNA) comprising an open reading frame (ORF) encoding a protein of interest, wherein the ORF is flanked by a heterologous 5′ untranslated region (UTR) and/or a heterologous 3′ UTR, wherein the 5′ UTR and/or 3′ UTR is derived from alpha-1-microglobulin/bikunin precursor (AMBP) or human alpha-globin gene.

2. The mRNA of claim 1, wherein the 5′ UTR comprises a nucleotide sequence selected from the group consisting of SEQ ID NOs: 3 and 1.

3. The mRNA of claim 1, wherein the 3′ UTR comprises a nucleotide sequence selected from the group consisting of SEQ ID NOs: 2 and 4.

4. The mRNA of claim 1, wherein the 5′ UTR comprises a nucleotide sequence of SEQ ID NO: 3 and the 3′ UTR comprises a nucleotide sequence of SEQ ID NO: 2; the 5′ UTR comprises a nucleotide sequence of SEQ ID NO: 1 and the 3′ UTR comprises a nucleotide sequence of SEQ ID NO: 2; the 5′ UTR comprises a nucleotide sequence of SEQ ID NO: 1 and the 3′ UTR comprises a nucleotide sequence of SEQ ID NO: 4; or the 5′ UTR comprises a nucleotide sequence of SEQ ID NO: 3 and the 3′ UTR comprises a nucleotide sequence of SEQ ID NO: 4.

5. The mRNA of claim 1, wherein the protein of interest is selected from the group consisting of phenylalanine hydroxylase (PAH), methylmalonyl-CoA mutase (MMUT), interferon alpha, TNF-related apoptosis-inducing ligand, vascular adhesion protein 1, hepatocyte growth factor, G6PC, ABCB11, ABCB4, ASL1, ASS, Arg1, AGXT, OTC, CBS, GBE, GALE, HADH, MCCC1, ADAMTS13, SLC25A15, C2, F2, PROS1, SERPINA1, GALT1, ETFA, GCDH, CTNS, FAH, TAT, HPD, HSD17b4, SLC2A2, GALC, ABCC6, AHSG, PDC-E2, GAA, ATP8B1, MMACHC, GK, PCCA, PCCB and UGT1A1 or fragments thereof.

6. The mRNA of claim 1, wherein the protein of interest is selected from the group consisting of SEQ ID NOs. 5, 6, 9, and 11 to 17.

7. The mRNA of claim 1, which further comprises a 5′ cap and/or a poly A tail.

8. The mRNA of claim 1, which comprises a nucleotide sequence selected from the group consisting of:
a combination of SEQ ID NOs. 3, 5, and 2;
a combination of SEQ ID NOs. 1, 5, and 4;
a combination of SEQ ID NOs. 1, 5, and 2;
a combination of SEQ ID NOs. 3, 13, and 2;
a combination of SEQ ID NOs. 3, 14, and 2;
a combination of SEQ ID NOs. 3, 15, and 2; and
a combination of SEQ ID NOs. 3, 16, and 2.

9. A DNA for transcripting the mRNA of claim 1.

10. The DNA of claim 9, which further comprises a promoter.

11. The DNA of claim 10, wherein the promoter is an RNA polymerase promoter.

12. A method for synthesizing the mRNA of claim 1, which comprises
providing the DNA for transcripting the mRNA; and
conducting a transcription form the DNA.

13. The method of claim 12, which further comprises conducting a capping reaction.

14. A composition comprising the mRNA of claim 1 and a pharmaceutically acceptable carrier.

15. The composition of claim 14, wherein the pharmaceutically acceptable carrier is a liposome or lipid nanoparticle.

16. A method for expressing a protein of interest in a cell comprising introducing the mRNA of claim 1 to the cell.

17. The method of claim 16, wherein the cell is a liver cell.

18. A method for treating a liver disease in a subject in need of such treatment comprising administering the mRNA of claim 1 and optionally a pharmaceutically acceptable carrier to the subject.

19. The method of claim 18, wherein the pharmaceutically acceptable carrier is a liposome or lipid nanoparticle.

20. The method of claim 18, wherein the liver disease is selected from the group consisting of phenylketonuria (PKU), methylmalonic aciduria (MMA), acute hepatitis, chronic hepatitis, liver cirrhosis, cirrhosis, fatty liver, liver cancer, glycogen storage disease, progressive familial intrahepatic cholestasis 1 (PFIC1), progressive familial intrahepatic cholestasis 2 (PFIC2), progressive familial intrahepatic cholestasis 3 (PFIC3), adenylosuccinate lyase deficiency (ASLD), citrullinemia, arginase-1 deficiency, primary hyperoxaluria type 1 (PH1), ornithine transcarbamylase deficiency (OTCD), homocystinuria, pheylketonuria, glycogen storage disease type IV (GSDIV), galactose-1-phosphate uridylyltransferase deficiency (type I (GALTI), type II (GALTII) or type 3 (GALTIII)), long chain 3-hydroxyacyl-CoA dehydrogenase deficiency (LCHADD), 3-methylcrotonyl-CoA carboxylase deficiency (MCCC1 deficiency), MMA and homocystinuria type C (MMACHC), thrombotic thrombocytopenic purpura (TTP), hyperornithinemia-hyperammonemia-homocitrullinuria syndrome (HHH), complement component 2 deficiency (C2D), F2, protein S deficiency (caused by allelic variants of PROS1), alpha-1 antitrypsin deficiency (A1AT), glutaric academia 1 (GA-1), glutaric academia 2 (GA-2), cystinosis (CTNS), tyrosinemia, tyrosinemia type 3 (cause by allelic variants of HPD), D-bifunctional protein deficiency (DBP), Fanconi-Bickel syndrome (FBS), pseudoxanthoma elasticum (PXE), primary biliary cirrhosis, Pompe disease, glycerol kinase deficiency (GKD), proprionic acidemia (PA) and Crigler-Najjar syndrome (CN1).


SEQUENCE LISTING (19 sequences)

SEQ ID NO: 1 - RNA, 91 nt, synthetic construct
  source 1..91: mol_type = other RNA
agttcacagctgccctgttgcagggaggcggtggcccttctgttgctagaccgagcctgtgggatataccaaggcagaggagcccatagcc

SEQ ID NO: 2 - RNA, 112 nt, synthetic construct
  source 1..112: mol_type = other RNA
caactggccggtctgcaagtcagaggatggccagtgtctgtcccggggtcctgtggcaggcagcgccaagcaacctgggtccaaataaaaactaaattgtaaactcctgaaa

SEQ ID NO: 3 - RNA, 54 nt, synthetic construct
  source 1..54: mol_type = other RNA
gagaataaactagtattcttctggtccccacagactcagagagaacccgccacc

SEQ ID NO: 4 - RNA, 111 nt, synthetic construct
  source 1..111: mol_type = other RNA
gctggagcctcggtggcctagcttcttgccccttgggcctccccccagcccctcctccccttcctgcacccgtacccccgtggtctttgaataaagtctgagtgggcggca

SEQ ID NO: 5 - RNA, 1011 nt, synthetic construct
  source 1..1011: mol_type = other RNA
atggtgccctggttccccagaaccatccaggagctggacagatttgccaaccagatcctgagctatggagctgaccagctggatgctgaccaccctggcttcaaggaccctgtgtacagagccagaagaaagcagtttgctgacattgcctacaactacagacatggccagcccatccccagagtggagtacatggaggaggagaagaagacctggggcacagtgttcaagaccctgaagagcctgtacaagacccatgcctgctatgagtacaaccacatcttccctctgctggagaagtactgtggcttccatgaggacaacatcccacagctggaggatgtgagccagttcctgcagacctgcacaggcttcagactgagacctgtggctggcctgctgagcagcagagacttcctgggaggcctggccttcagagtgttccactgcacccagtacatcagacatggcagcaagcccatgtacacacctgagcctgacatctgccatgagctgctgggccatgtgcccctgttcagtgacagaagctttgcccagttcagccaggagattggcctggccagcctgggagcccctgatgagtacattgagaagctggccaccatctactggttcacagtggagtttggcctgtgcaagcagggagacagcatcaaggcctatggagctggcctgctgagcagctttggagagctgcagtactgcctgagtgagaagcccaagctgctgcccctggagctggagaagacagccatccagaactacacagtgacagagttccagcccctgtactatgtggctgagagcttcaatgatgccaaggagaaggtgagaaactttgctgccaccatccccagacccttcagtgtgagatatgacccctacacccagagaattgaggtgctggacaacacccagcagctgaagatcctggctgacagcatcaacagtgagattggcatcctgtgcagtgccctgcagaagatcaag

SEQ ID NO: 6 - RNA, 1356 nt, synthetic construct
  source 1..1356: mol_type = other RNA
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaag

SEQ ID NO: 7 - RNA, 47 nt, synthetic construct
  source 1..47: mol_type = other RNA
gggaaataagagagaaaagaagagtaagaagaaatataagagccacc

SEQ ID NO: 8 - RNA, 133 nt, synthetic construct
  source 1..133: mol_type = other RNA
gctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 9 - RNA, 1055 nt, synthetic construct
  source 1..1055: mol_type = other RNA
gggaaataagagagaaaagaagagtaagaagaaatataagagccaccatggtgccctggttcccacggaccatccaggagctggaccggttcgccaaccagatcctgagctacggcgccgagctggacgccgaccaccccggcttcaaggaccccgtgtaccgggcccggcggaagcagttcgccgacatcgcctacaactaccggcacggccagcccatcccgcgggtggagtacatggaggaggagaagaagacctggggcaccgtgttcaagaccctgaagagcctgtacaagacccacgcctgctacgagtacaaccacatcttccctctgctggagaagtactgcggcttccacgaggacaacatcccgcagctggaggacgtgagccagttcctgcagacctgcaccggcttccggctgcggcccgtggccggcctgctgagcagccgggacttcctgggcggcctggccttccgggtgttccactgcacccagtacatccggcacggcagcaagcccatgtacacgcccgagcccgacatctgccacgagctgctgggccacgtgcccctgttcagcgaccggagcttcgcccagttcagccaggagatcggcctggccagcctgggcgcgcccgacgagtacatcgagaagctggccaccatctactggttcaccgtggagttcggcctgtgcaagcagggcgacagcatcaaggcctacggcgccggcctgctgagcagcttcggcgagctgcagtactgcctgagcgagaagcccaagctgctgcccctggagctggagaagaccgccatccagaactacaccgtgaccgagttccagcccctgtactacgtggccgagagcttcaacgacgccaaggagaaggtgcggaacttcgccgccaccatccctcggcccttcagcgtgcggtacgacccctacacccagcggatcgaggtgctggacaacacccagcagctgaagatcctggccgacagcatcaacagcgagatcggcatcctgtgcagcgccctgcagaagatcaag

SEQ ID NO: 10 - RNA, 132 nt, synthetic construct
  source 1..132: mol_type = other RNA
gctggagcctcggtggcctagcttcttgccccttgggcctccccccagcccctcctccccttcctgcacccgtaccccccgcattattactcacggtacgagtggtctttgaataaagtctgagtgggcggc

SEQ ID NO: 11 - RNA, 1356 nt, synthetic construct
  source 1..1356: mol_type = other RNA
atgagcaccgccgtgctggagaaccccggcctgggccggaagctgagcgacttcggccaggagacaagctacatcgaggacaactgcaaccagaacggcgccatcagcctgatcttttctctgaaggaggaggtgggcgccctggccaaggtgctgcggctgttcgaggagaacgacgtgaacctgacccacatcgagagccggcccagccggctgaagaaggacgagtacgagttcttcacccacctggacaagcggagcctgcccgccctgaccaacatcatcaagatcctgcggcacgacatcggcgccaccgtgcacgagctgagccgggacaagaagaaggacaccgtgccctggttccctcggaccatccaggagctggaccggttcgccaaccagatcctgagctacggcgccgagctggacgccgaccaccccggcttcaaggaccccgtgtaccgggcccggcggaagcagttcgccgacatcgcctacaactaccggcacggccagcccatccctcgggtggagtacatggaggaggagaagaagacctggggcaccgtgttcaagaccctgaagtctctgtacaagacccacgcctgctacgagtacaaccacatctttcctctcctggagaagtactgcggcttccacgaggacaatatccctcagctggaggacgtgagccagttcctgcagacctgcaccggcttccggctgaggcctgtggccgggctgctgagcagcagagacttcctgggcggcctggccttccgggtgttccactgcacccagtacatcagacacgggagcaagcccatgtacactcccgagcccgacatctgccacgagttactgggccacgtgcccctgttcagcgaccggagcttcgcccagttctcacaggagatcgggctggcaagcctgggcgctcccgacgagtatatagagaagctggccaccatctactggttcaccgtggagttcggcctgtgcaagcagggcgacagcatcaaggcttacggagctgggctgcttagctccttcggcgagctgcagtactgcctgagcgagaagcccaagctgctgccccttgagctcgagaagaccgccatccagaactacaccgtgaccgagttccagcccctgtactacgtggccgagagcttcaacgacgccaaggagaaggtgcggaacttcgccgcaaccatccctaggcccttcagcgtgcggtacgacccctacacccagcggatcgaggtgctggacaatacccagcagctgaagatcttagctgactcaatcaacagcgagattggcatcctgtgcagcgccctgcagaagatcaag

SEQ ID NO: 12 - RNA, 1008 nt, synthetic construct
  source 1..1008: mol_type = other RNA
atggtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaag

SEQ ID NO: 13 - RNA, 2250 nt, synthetic construct
  source 1..2250: mol_type = other RNA
atgctgagagccaagaaccagctgttcctgctgagccctcactacctgagacaggtgaaggagagcagcggcagcagactgatccagcagagactgctgcaccagcagcagcccctgcaccccgagtgggccgccctggccaagaagcagctgaagggcaagaaccccgaggacctgatctggcacacgcccgagggcatcagcatcaagcccctgtacagcaagagagacaccatggacctgcccgaggagctgcccggcgtgaagcccttcaccagaggcccctaccccaccatgtacaccttcagaccctggaccatcagacagtacgccggcttcagcaccgtggaggagagcaacaagttctacaaggacaacatcaaggccggccagcagggcctgagcgtggccttcgacctggccacccacagaggctacgacagcgacaaccccagagtgagaggcgacgtgggcatggccggcgtggccatcgacaccgtggaggacaccaagatcctgttcgacggcatccctctggagaagatgagcgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgagaaacacctacatcttccctcccgagcccagcatgaagatcatcgccgacatcttcgagtacaccgccaagcacatgcccaagttcaacagcatcagcatcagcggctaccacatgcaggaggccggcgccgacgccatcctggagctggcctacaccctggccgacggcctggagtacagcagaaccggcctgcaggccggcctgaccatcgacgagttcgcgcccagactgagcttcttctggggcatcggcatgaacttctacatggagatcgccaagatgagagccggcagaagactgtgggcccacctgatcgagaagatgttccagcccaagaacagcaagagcctgctgctgagagcccactgccagaccagcggctggagcctgaccgagcaggacccctacaacaacatcgtgagaaccgccatcgaggccatggccgccgtgttcggcggcacccagagcctgcacaccaacagcttcgacgaggccctgggcctgcccaccgtgaagagcgccagaatcgccagaaacacccagatcatcatccaggaggagagcggcatccccaaggtggccgacccctggggcggcagctacatgatggagtgcctgaccaacgacgtgtacgacgccgccctgaagctgatcaacgagatcgaggagatgggcggcatggccaaggccgtggccgagggcatccccaagctgagaatcgaggagtgcgccgccagaagacaggccagaatcgacagcggcagcgaggtgatcgtgggcgtgaacaagtaccagctggagaaggaggacgccgtggaggtgctggccatcgacaacaccagcgtgagaaacagacagatcgagaagctgaagaagatcaagagcagcagagaccaggccctggccgagagatgcctggccgccctgaccgagtgcgccgccagcggcgacggcaacatcctggccctggccgtggacgccagcagagccagatgcaccgtgggcgagatcaccgacgccctgaagaaggtgttcggcgagcacaaggccaacgacagaatggtgagcggcgcctacagacaggagttcggcgagagcaaggagatcaccagcgccatcaagagagtgcacaagttcatggagagagagggcagaagacccagactgctggtggccaagatgggccaggacggccacgacagaggcgccaaggtgatcgccaccggcttcgccgacctgggcttcgacgtggacatcggcccactgttccagacgcccagagaggtggcccagcaggccgtggacgccgacgtgcacgccgtgggcatcagcaccctggccgccggccacaagaccctggtgcccgagctgatcaaggagctgaacagcctgggcagacccgacatcctggtgatgtgcggcggcgtgatcccgccccaggactacgagttcctgttcgaggtgggcgtgagcaacgtgttcggccccggcaccagaatccccaaggccgccgtgcaggtgctggacgacatcgagaagtgcctggagaagaagcagcagagcgtg

SEQ ID NO: 14 - RNA, 2250 nt, synthetic construct
  source 1..2250: mol_type = other RNA
atgctgagagccaagaaccagctgttcctgctgagccctcactacctgagacaggtgaaggagagcagtggcagcagactgatccagcagagactgctgcaccagcagcagcccctgcaccctgagtgggctgccctggccaagaagcagctgaagggcaagaaccctgaggacctgatctggcacacacctgagggcatcagcatcaagcccctgtacagcaagagagacaccatggacctgcctgaggagctgcctggggtgaagcccttcaccagaggcccctaccccaccatgtacaccttcagaccctggaccatcagacagtatgctggcttcagcacagtggaggagagcaacaagttctacaaggacaacatcaaggctggccagcagggcctgagtgtggcctttgacctggccacccacagaggctatgacagtgacaaccccagagtgagaggggatgtgggcatggctggggtggccattgacacagtggaggacaccaagatcctgtttgatggcatccctctggagaagatgagtgtgagcatgaccatgaatggggctgtgatccctgtgctggccaacttcattgtgacaggggaggagcagggggtgcccaaggagaagctgacaggcaccatccagaatgacatcctgaaggagttcatggtgagaaacacctacatcttccctcctgagcccagcatgaagatcattgctgacatctttgagtacacagccaagcacatgcccaagttcaacagcatcagcatcagtggctaccacatgcaggaggctggggctgatgccatcctggagctggcctacaccctggctgatggcctggagtacagcagaacaggcctgcaggctggcctgaccattgatgagtttgctcccagactgagcttcttctggggcattggcatgaacttctacatggagattgccaagatgagagctggcagaagactgtgggcccacctgattgagaagatgttccagcccaagaacagcaagagcctgctgctgagagcccactgccagaccagtggctggagcctgacagagcaggacccctacaacaacattgtgagaacagccattgaggccatggctgctgtgtttgggggcacccagagcctgcacaccaacagctttgatgaggccctgggcctgcccacagtgaagagtgccagaattgccagaaacacccagatcatcatccaggaggagagtggcatccccaaggtggctgacccctgggggggcagctacatgatggagtgcctgaccaatgatgtgtatgatgctgccctgaagctgatcaatgagattgaggagatggggggcatggccaaggctgtggctgagggcatccccaagctgagaattgaggagtgtgctgccagaagacaggccagaattgacagtggcagtgaggtgattgtgggggtgaacaagtaccagctggagaaggaggatgctgtggaggtgctggccattgacaacaccagtgtgagaaacagacagattgagaagctgaagaagatcaagagcagcagagaccaggccctggctgagagatgcctggctgccctgacagagtgtgctgccagtggggatggcaacatcctggccctggctgtggatgccagcagagccagatgcacagtgggggagatcacagatgccctgaagaaggtgtttggggagcacaaggccaatgacagaatggtgagtggggcctacagacaggagtttggggagagcaaggagatcaccagtgccatcaagagagtgcacaagttcatggagagagagggcagaagacccagactgctggtggccaagatgggccaggatggccatgacagaggggccaaggtgattgccacaggctttgctgacctgggctttgatgtggacattggcccactgttccagacacccagagaggtggcccagcaggctgtggatgctgatgtgcatgctgtgggcatcagcaccctggctgctggccacaagaccctggtgcctgagctgatcaaggagctgaacagcctgggcagacctgacatcctggtgatgtgtgggggggtgatccctccccaggactatgagttcctgtttgaggtgggggtgagcaatgtgtttggccctggcaccagaatccccaaggctgctgtgcaggtgctggatgacattgagaagtgcctggagaagaagcagcagagtgtg

SEQ ID NO: 15 - RNA, 2250 nt, synthetic construct
  source 1..2250: mol_type = other RNA
atgctgcgggccaagaaccagctgttcctgctgagccctcactacctgagacaggtgaaggagagcagcggcagcagactgatccagcagagactgctgcaccagcagcagcccctgcaccccgagtgggccgccctggccaagaagcagctgaagggcaagaaccccgaggacctgatctggcacacgcccgagggcatcagcatcaagcccctgtacagcaagcgggacaccatggacctgcccgaggagctgcccggcgtgaagcccttcacccggggcccctaccccaccatgtacaccttcagaccctggaccatcagacagtacgccggcttcagcaccgtggaggagagcaacaagttctacaaggacaacatcaaggccggccagcagggcctgagcgtggccttcgacctggccacccacagaggctacgacagcgacaacccacgggtgagaggcgacgtgggcatggccggcgtggccatcgacaccgtggaggacaccaagatcctgttcgacggcatccctctggagaagatgagcgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgagaaacacctacatcttccctcccgagcccagcatgaagatcatcgccgacatcttcgagtacaccgccaagcacatgcccaagttcaacagcatcagcatcagcggctaccacatgcaggaggccggcgccgacgccatcctggagctggcctacaccctggccgacggcctggagtacagccggaccggcctgcaggccggcctgaccatcgacgagttcgcgcccagactgagcttcttctggggcatcggcatgaacttctacatggagatcgccaagatgagagccggcagaagactgtgggcccacctgatcgagaagatgttccagcccaagaacagcaagagcctgctgctgagagcccactgccagaccagcggctggagcctgaccgagcaggacccctacaacaacatcgtgagaaccgccatcgaggccatggccgccgtgttcggcggcacccagagcctgcacaccaacagcttcgacgaggccctgggcctgcccaccgtgaagagcgcccggatcgcccggaacacccagatcatcatccaggaggagagcggcatccccaaggtggccgacccctggggcggcagctacatgatggagtgcctgaccaacgacgtgtacgacgccgccctgaagctgatcaacgagatcgaggagatgggcggcatggccaaggccgtggccgagggcatccccaagctgagaatcgaggagtgcgccgcccggagacaggccagaatcgacagcggcagcgaggtgatcgtgggcgtgaacaagtaccagctggagaaggaggacgccgtggaggtgctggccatcgacaacaccagcgtgagaaacagacagatcgagaagctgaagaagatcaagagcagcagagaccaggccctggccgagcggtgcctggccgccctgaccgagtgcgccgccagcggcgacggcaacatcctggccctggccgtggacgccagcagagcccggtgcaccgtgggcgagatcaccgacgccctgaagaaggtgttcggcgagcacaaggccaacgaccggatggtgagcggcgcctacagacaggagttcggcgagagcaaggagatcaccagcgccatcaagagagtgcacaagttcatggagagagagggccggcggccccggctgctggtggccaagatgggccaggacggccacgacagaggcgccaaggtgatcgccaccggcttcgccgacctgggcttcgacgtggacatcggcccactgttccagacgcccagagaggtggcccagcaggccgtggacgccgacgtgcacgccgtgggcatcagcaccctggccgccggccacaagaccctggtgcccgagctgatcaaggagctgaacagcctgggccggcccgacatcctggtgatgtgcggcggcgtgatcccgccccaggactacgagttcctgttcgaggtgggcgtgagcaacgtgttcggccccggcaccagaatccccaaggccgccgtgcaggtgctggacgacatcgagaagtgcctggagaagaagcagcagagcgtg

SEQ ID NO: 16 - RNA, 2250 nt, synthetic construct
  source 1..2250: mol_type = other RNA
atgttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgta

SEQ ID NO: 17 - RNA, 2250 nt, synthetic construct
  source 1..2250: mol_type = other RNA
atgctgagggccaagaaccagctgttcctgctgagtccccactacctgcggcaggtgaaggagagcagcggcagccggctgatccagcagcggctgctgcaccagcagcagcccctgcaccccgagtgggccgccctggccaagaagcagctgaagggcaagaaccccgaggacctgatctggcacacacccgagggcatcagcatcaagcccctgtacagcaagagggacaccatggacctgcctgaggagctgcccggcgtgaagcccttcacccgcgggccctaccccaccatgtacaccttccggccctggaccatccggcagtacgccgggttctccaccgtggaggagtccaacaagttctacaaggacaacatcaaggccgggcagcagggcctgtccgtggccttcgacctggccacccaccggggctacgacagcgacaatccccgcgtgcggggcgacgtgggcatggccggcgtggccatcgataccgtggaggacaccaagatcctgttcgacgggatcccactggagaagatgtccgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgcggaacacctacatcttcccacccgagcccagcatgaagatcatcgccgacatcttcgagtacaccgccaagcacatgcccaagttcaactccatcagcatcagcggctaccacatgcaggaggccggagccgacgccatcctggagctggcctacaccctcgccgacggcctggagtacagccgcaccggcctgcaagccggcctgaccatcgacgagttcgctccccggctgagcttcttctggggcatcgggatgaacttctacatggagatcgccaagatgcgggccggacggcggctgtgggcccacctgatcgagaagatgttccagcccaagaacagcaagtccctgctgctgcgggcccactgccagacctccggctggagcctgaccgagcaggacccctacaacaacatcgtgcggaccgccatcgaggccatggccgccgtgttcggcggcacccagagcctgcacaccaacagcttcgacgaggccctgggcctgcccaccgtgaagtccgcccgcatcgcccgcaacacccagatcatcatccaggaggagtccggcatccccaaggtggccgacccctggggcggcagctacatgatggagtgcctgaccaacgacgtgtacgacgccgccctgaagctgatcaacgagatcgaggagatgggcgggatggccaaggccgtggccgagggcattcccaagctgcggatcgaggagtgtgccgccaggcggcaggctcggatcgactccggctccgaggtgatcgtgggcgtgaacaagtaccagctggagaaggaggacgccgtggaggtgctggccatcgacaacaccagcgtgcggaaccggcagatcgagaagctgaagaagatcaagagcagccgggaccaggccctggccgagaggtgcctggccgccctgaccgagtgcgccgcttcgggcgacggcaacatcctggccttagccgtggacgcaagccgggccaggtgcaccgtgggcgagatcaccgacgccctgaagaaggtgttcggcgagcacaaggccaacgaccgcatggtgtccggcgcctaccggcaggagttcggcgagagcaaggagatcaccagcgccatcaagcgggtgcacaagttcatggaacgggagggccgcagaccccgcctgctggtggccaagatgggccaggacggccacgaccggggcgccaaggtgatcgccaccgggttcgccgacctgggcttcgacgtggacatcggacccctgttccagaccccacgggaggtggcccagcaggccgtggacgccgacgtgcacgccgtgggcatctccaccctggccgccgggcacaagaccctggtgcccgagctgatcaaggagctgaactccctgggccgccccgacatcctggtgatgtgcggcggcgtgatcccaccccaggactacgagttcctgttcgaggtgggcgtgagcaacgtgttcggccccggcacccggatccctaaggccgccgtgcaggtgctggacgacatcgagaagtgcctggagaagaagcagcagtccgtg

SEQ ID NO: 18 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 19 - RNA, 241 nt, synthetic construct
  source 1..241: mol_type = other RNA
taatagtaaacctcactcacggccacattgagtgccaggctccgggctggtttatagtagtgtagagcattgcagcacttagactggggtgctgtagtctttattgtagtctttccacatacctgataattcttagataatttcttattttaattccataaagtaggaaacactacataaatctccataaagtaggaaacactacatattcttccataaagtaggaaacactacataggct